(19) United States
(12) Patent Application Publication
(10) Pub. No.: US 2023/0136188 A1
(43) Pub. Date: May 4, 2023

(54) Glycemic Impact Prediction For Improving Diabetes Management

(71) Applicant: Dexcom, Inc., San Diego, CA (US)

(72) Inventors: Sarah Kate Pickus, San Diego, CA (US); Margaret A. Crawford, San Diego, CA (US); Mark Derdzinski, San Diego, CA (US); Lauren H. Jepson, San Diego, CA (US); Robert J. Dowd, San Diego, CA (US); Giada Acciaroli, San Diego, CA (US); Apurv U. Kamath, San Diego, CA (US)

(73) Assignee: Dexcom, Inc., San Diego, CA (US)

(21) Appl. No.: 17/974,296

(22) Filed: Oct. 26, 2022

Related U.S. Application Data
(60) Provisional application No. 63/263,188, filed on Oct. 28, 2021.
(60) Provisional application No. 63/292,979, filed on Dec. 22, 2021.

(51) Int. Cl. A61B 5/00; A61B 5/145; A61B 5/11

(52) U.S. Cl. CPC A61B 5/486; A61B 5/742; A61B 5/1118; A61B 5/4806; A61B 5/4866; A61B 5/7267; A61B 5/7275; A61B 5/14532

(57) ABSTRACT

Glucose level measurements and additional data regarding a user are obtained over time, such as from a wearable glucose monitoring device being worn by the user. This additional data identifies events or conditions that may affect glucose of the user, such as physical activity engaged in by the user. A glucose prediction system analyzes, for example, activity data of the user and determines when a bout of physical activity occurs. The glucose prediction system predicts what the glucose measurements of the user would have been had the physical activity not occurred, and takes various actions based on the predicted glucose measurements (e.g., provides feedback to the user indicating what their glucose would have been had they not engaged in the physical activity).

### RELATED APPLICATIONS

This application claims the benefit of U.S. Provisional Pat. Application No. 63/292,979, filed Dec. 22, 2021, and titled “Glycemic Impact Prediction For Improving Diabetes Management,” the entire disclosure of which is hereby incorporated by reference, and also claims the benefit of U.S. Provisional Pat. Application No. 63/263,188, filed Oct. 28, 2021, and titled “Ranking Feedback For Improving Diabetes Management,” the entire disclosure of which is hereby incorporated by reference.

### BACKGROUND

Diabetes is a metabolic condition affecting hundreds of millions of people and is one of the leading causes of death worldwide. For people living with Type I diabetes, access to treatment is critical to their survival and it can reduce adverse outcomes among people with Type II diabetes. With proper treatment, serious damage to the heart, blood vessels, eyes, kidneys, and nerves due to diabetes can be avoided. Regardless of the type of diabetes (e.g., Type I or Type II), managing diabetes successfully involves monitoring and oftentimes adjusting food and activity to control a person’s blood glucose, such as to reduce severe fluctuations in and/or generally lower the person’s glucose.

However, many conventional glucose monitoring applications employ user interfaces that display raw glucose information in a manner that is difficult for users to interpret, particularly users who have just recently started monitoring their glucose. Consequently, users may be unable to draw insights from the data and thus are unable to alter their behavior in a meaningful way in order to improve their glucose. Over time, these users often become overwhelmed and frustrated by the manner in which information is presented by these conventional glucose monitoring applications and thus discontinue use of these applications before improvements in their glucose and overall health can be realized. Moreover, as users increasingly utilize mobile devices (e.g., smart watches and smart phones) to access glucose monitoring information, the failure by conventional systems to provide meaningful glucose information in a manner that users can understand is further exacerbated by the constraints imposed by the small screens of these mobile devices.

### SUMMARY

To overcome these problems, techniques for glycemic impact prediction for improving diabetes management are discussed. In one or more implementations, in a continuous glucose level monitoring system, glucose measurements measured for a user are obtained from a glucose sensor of the continuous glucose level monitoring system, the glucose sensor being inserted at an insertion site of the user. A bout of physical activity performed by the user is detected and an impact of the physical activity on glucose of the user is predicted. This impact is predicted by generating, based on the glucose measurements, one or more predicted glucose measurements that the user would have had if, during a time the user was performing the bout of physical activity, the user had not performed the bout of physical activity. The one or more predicted glucose measurements are caused to be displayed.

In one or more implementations, in a diabetes management monitoring system, glucose measurements measured for a user are obtained, from a sensor of the diabetes management monitoring system. A determination is made that a bout of physical activity was not performed by the user during a duration of time. An impact of not performing the physical activity on glucose of the user is predicted by generating, based on the glucose measurements, one or more predicted glucose measurements that the user would have had if, during a time the user was performing the bout of physical activity, the user had performed the bout of physical activity. The one or more predicted glucose measurements are caused to be displayed.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures.

FIG. 1 is an illustration of an environment in an example of an implementation that is operable to implement glycemic impact prediction for improving diabetes management as described herein.

FIG. 2 depicts an example of an implementation of a wearable glucose monitoring device in greater detail.

FIG. 3 is an illustration of an example architecture of a glucose prediction system.

FIG. 4 illustrates an example of generating predicted glucose measurements.

FIG. 5 illustrates an example of providing predicted glucose measurements.

FIG. 6 depicts a procedure in an example of implementing glycemic impact prediction for improving diabetes management.

FIG. 7 depicts a procedure in another example of implementing glycemic impact prediction for improving diabetes management.

FIG. 8 illustrates an example of a system that includes an example of a computing device that is representative of one or more computing systems and/or devices that may implement the various techniques described herein.

### DETAILED DESCRIPTION

### Overview

Techniques for glycemic impact prediction for improving diabetes management are discussed herein. Broadly, blood glucose level measurements of a user are obtained over time. Glucose level measurements are typically obtained by a wearable glucose monitoring device being worn by the user. These glucose level measurements can be produced substantially continuously, such that the device may be configured to produce the glucose level measurements at regular or irregular intervals of time (e.g., approximately every hour, approximately every 30 minutes, approximately every 5 minutes, and so forth), responsive to establishing a communicative coupling with a different device (e.g., when a computing device establishes a wireless connection with a wearable glucose level monitoring device to retrieve one or more of the measurements), and so forth.

In one or more implementations, a data stream of glucose measurements is received. Various other data streams are also received, such as activity data (e.g., number of steps taken by the user). A glucose prediction system analyzes, for example, activity data of a user and determines when a bout of physical activity occurs. The glucose prediction system predicts what the glucose measurements of the user would have been had the physical activity not occurred, and takes various actions based on the predicted glucose measurements (e.g., provides feedback to the user indicating what their glucose would have been had they not engaged in the physical activity).

Additionally or alternatively, the received data streams include various other data that indicates events or conditions that may affect glucose of the user, such as activities the user engages in, behaviors of the user, reactions of the user, medical conditions of the user, biological data of the user, and so forth. The glucose prediction system analyzes this data to identify such events or conditions, and predicts what the glucose measurements of the user would have been had the identified events or conditions not occurred or not been present. The glucose prediction system takes various actions based on the predicted glucose measurements (e.g., provides feedback to the user indicating what their glucose would have been had the identified events or conditions not occurred or not been present).

The techniques discussed herein apply analogously to determining when a period of physical activity does not occur (or other events or conditions do not occur or are not present). The glucose prediction system predicts what the glucose measurements of the user would have been had the physical activity occurred (or other events or conditions occurred or been present), and takes various actions based on the predicted glucose measurements (e.g., provides feedback to the user indicating what their glucose would have been had they engaged in the physical activity or if other events or conditions had occurred or been present).

The techniques discussed herein predict or estimate what glucose measurements would have been for a user if particular events or conditions had occurred or been present (or had not occurred or been present). Feedback giving positive reinforcement of one or both of healthy glucose management behavior modifications and patient-specific goals (e.g., using activity to mitigate post-prandial spikes or lower blood glucose after sustained hyperglycemia) is provided. This helps the user improve diabetes management and his or her overall health.

Furthermore, the techniques discussed herein provide real-time teachable moments by linking specific behavior modifications to improved diabetes management outcomes. The user receives real-time feedback allowing the user to know that his behavior or choices have had a positive impact on his glucose, allowing him to continue such behavior in the future and improve his overall health.

In the following discussion, an example environment is first described that may employ the techniques described herein. Examples of implementation details and procedures are then described which may be performed in the example environment as well as other environments. Performance of the example procedures is not limited to the example environment and the example environment is not limited to performance of the example procedures.

### Example of an Environment

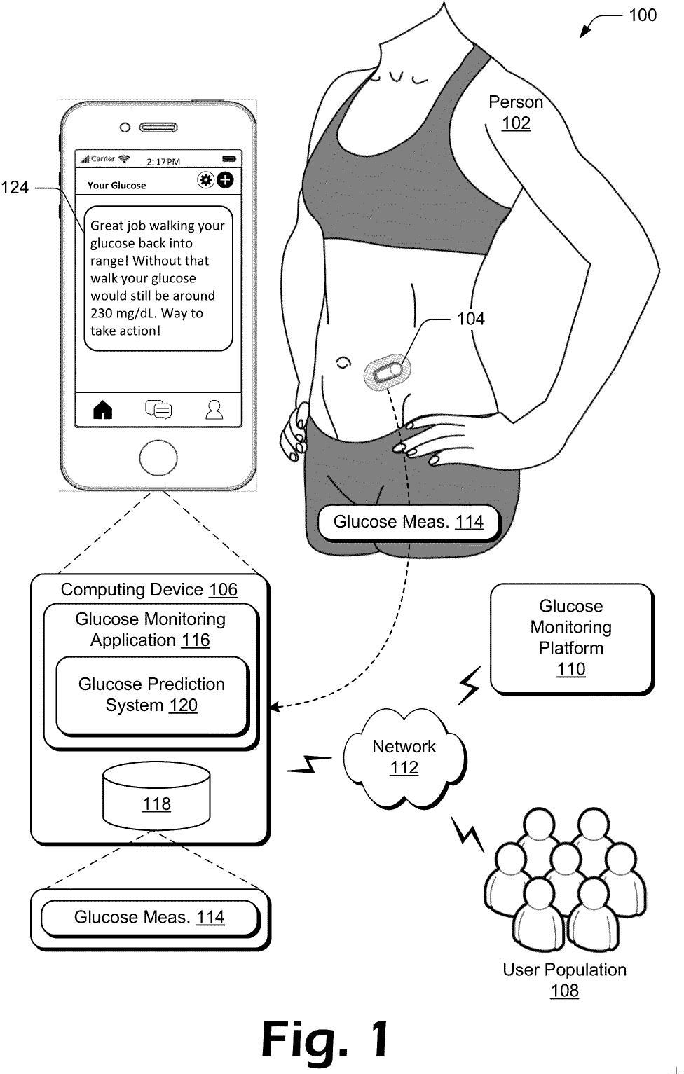

FIG. 1 is an illustration of an environment 100 in an example of an implementation that is operable to implement glycemic impact prediction for improving diabetes management as described herein. The illustrated environment 100 includes a person 102, who is depicted wearing a wearable glucose monitoring device 104. The illustrated environment 100 also includes a computing device 106, other users in a user population 108 that wear glucose monitoring devices 104, and a glucose monitoring platform 110. The wearable glucose monitoring device 104, computing device 106, user population 108, and glucose monitoring platform 110 are communicatively coupled, including via a network 112.

Alternately or additionally, the wearable glucose monitoring device 104 and the computing device 106 may be communicatively coupled in other ways, such as using one or more wireless communication protocols or techniques. By way of example, the wearable glucose monitoring device 104 and the computing device 106 may communicate with one another using one or more of Bluetooth (e.g., Bluetooth Low Energy links), near-field communication (NFC), 5G, and so forth.

In accordance with the described techniques, the wearable glucose monitoring device 104 is configured to provide measurements of person 102’s glucose. These measurements indicate the person 102’s glucose levels. Although a wearable glucose monitoring device is discussed herein, it is to be appreciated that user interfaces for glucose monitoring may be generated and presented in connection with other devices capable of providing glucose measurements, e.g., non-wearable glucose devices such as blood glucose meters requiring finger sticks, patches, and so forth. In implementations that involve the wearable glucose monitoring device 104, though, it may be configured with a glucose sensor that continuously detects analytes indicative of the person 102’s glucose and enables generation of glucose measurements. In the illustrated environment 100 and throughout the detailed description these measurements are represented as glucose measurements 114.

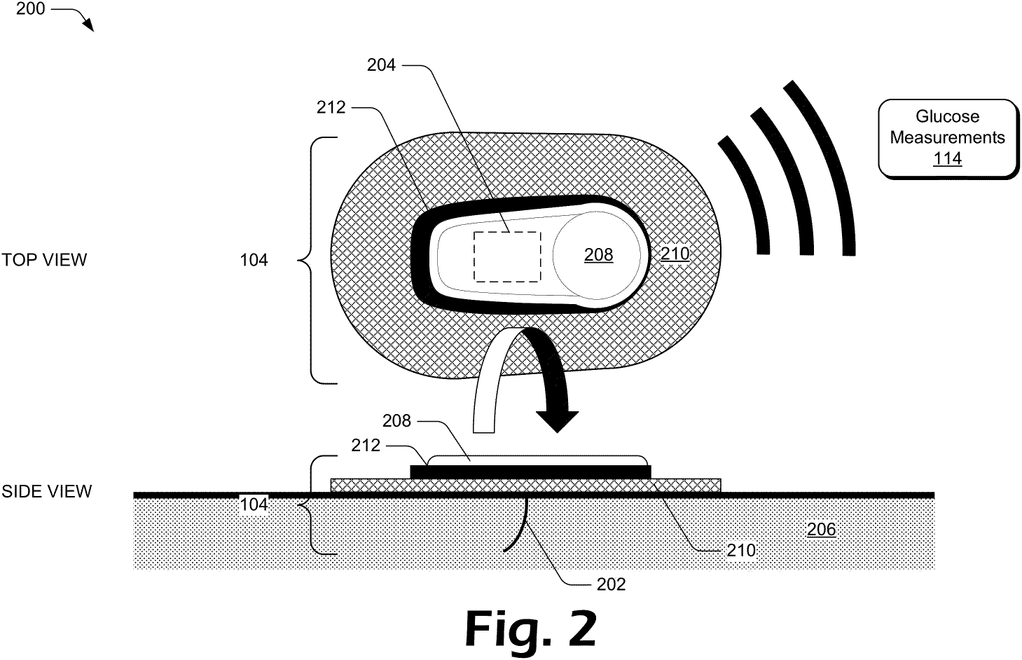

In one or more implementations, the wearable glucose monitoring device 104 is a continuous glucose monitoring (“CGM”) system. As used herein, the term “continuous” used in connection with glucose monitoring may refer to an ability of a device to produce measurements substantially continuously, such that the device may be configured to produce the glucose measurements 114 at regular or irregular intervals of time (e.g., every hour, every 30 minutes, every 5 minutes, and so forth), responsive to establishing a communicative coupling with a different device (e.g., when a computing device establishes a wireless connection with the wearable glucose monitoring device 104 to retrieve one or more of the measurements), and so forth. This functionality along with further aspects of the wearable glucose monitoring device 104’s configuration is discussed in more detail in relation to FIG. 2.

Additionally, the wearable glucose monitoring device 104 transmits the glucose measurements 114 to the computing device 106, such as via a wireless connection. The wearable glucose monitoring device 104 may communicate these measurements in real-time, e.g., as they are produced using a glucose sensor. Alternately or in addition, the wearable glucose monitoring device 104 may communicate the glucose measurements 114 to the computing device 106 at set time intervals. For example, the wearable glucose monitoring device 104 may be configured to communicate the glucose measurements 114 to the computing device 106 every five minutes (as they are being produced).

Certainly, an interval at which the glucose measurements 114 are communicated may be different from the examples above without departing from the spirit or scope of the described techniques. The measurements may be communicated by the wearable glucose monitoring device 104 to the computing device 106 according to other bases in accordance with the described techniques, such as based on a request from the computing device 106. Regardless, the computing device 106 may maintain the glucose measurements 114 of the person 102 at least temporarily, e.g., in computer-readable storage media of the computing device 106.

Although illustrated as a mobile device (e.g., a mobile phone), the computing device 106 may be configured in a variety of ways without departing from the spirit or scope of the described techniques. By way of example and not limitation, the computing device 106 may be configured as a different type of device, such as a mobile device (e.g., a wearable device, tablet device, or laptop computer), a stationary device (e.g., a desktop computer), an automotive computer, and so forth. In one or more implementations, the computing device 106 may be configured as a dedicated device associated with the glucose monitoring platform 110, e.g., with functionality to obtain the glucose measurements 114 from the wearable glucose monitoring device 104, perform various computations in relation to the glucose measurements 114, display information related to the glucose measurements 114 and the glucose monitoring platform 110, communicate the glucose measurements 114 to the glucose monitoring platform 110, and so forth.

Additionally, the computing device 106 may be representative of more than one device in accordance with the described techniques. In one or more scenarios, for instance, the computing device 106 may correspond to both a wearable device (e.g., a smart watch) and a mobile phone. In such scenarios, both of these devices may be capable of performing at least some of the same operations, such as to receive the glucose measurements 114 from the wearable glucose monitoring device 104, communicate them via the network 112 to the glucose monitoring platform 110, display information related to the glucose measurements 114, and so forth. Alternately or in addition, different devices may have different capabilities that other devices do not have or that are limited through computing instructions to specified devices.

In the scenario where the computing device 106 corresponds to a separate smart watch and a mobile phone, for instance, the smart watch may be configured with various sensors and functionality to measure a variety of physiological markers (e.g., heartrate, heartrate variability, breathing, rate of blood flow, and so on) and activities (e.g., steps or other exercise) of the person 102. In this scenario, the mobile phone may not be configured with these sensors and functionality, or it may include a limited amount of that functionality—although in other scenarios a mobile phone may be able to provide the same functionality. Continuing with this particular scenario, the mobile phone may have capabilities that the smart watch does not have, such as a camera to capture images associated with glucose monitoring and an amount of computing resources (e.g., battery and processing speed) that enables the mobile phone to more efficiently carry out computations in relation to the glucose measurements 114. Even in scenarios where a smart watch is capable of carrying out such computations, computing instructions may limit performance of those computations to the mobile phone so as not to burden both devices and to utilize available resources efficiently. To this extent, the computing device 106 may be configured in different ways and represent different numbers of devices than discussed herein without departing from the spirit and scope of the described techniques.

In accordance with the discussed techniques, the computing device 106 is configured to implement glycemic impact prediction for improving diabetes management. In the environment 100, the computing device 106 includes glucose monitoring application 116 and storage device 118. Here, the glucose monitoring application 116 includes the glucose prediction system 120. Although illustrated as being included in computing device 106, additionally or alternatively at least some functionality of the glucose prediction system 120 is located elsewhere, such as in glucose monitoring platform 110. Further, the glucose measurements 114 are shown stored in the storage device 118. The storage device 118 may represent one or more databases and also other types of storage capable of storing the glucose measurements 114.

In one or more implementations, the glucose measurements 114 may be stored at least partially remote from the computing device 106, e.g., in storage of the glucose monitoring platform 110, and retrieved or otherwise accessed in connection with configuring and outputting (e.g., displaying) user interfaces for diabetes management feedback presentation. For instance, the glucose measurements 114 may be generally stored in storage of the glucose monitoring platform 110 along with the glucose measurements of the user population 108, and some of that data may be retrieved or otherwise accessed on an as-needed basis to display user interfaces for diabetes management feedback presentation.

Broadly speaking, the glucose monitoring application 116 is configured to support interactions with a user that enable feedback about the user’s glucose and predicted glucose to be presented. This may include, for example, obtaining the glucose measurements 114 for processing (e.g., to determine the appropriate feedback), receiving information about a user (e.g., through an onboarding process and/or user feedback), causing information to be communicated to a health care provider, causing information to be communicated to the glucose monitoring platform 110, and so forth.

In one or more implementations, the glucose monitoring application 116 also leverages resources of the glucose monitoring platform 110 in connection with glycemic impact prediction for improving diabetes management. As noted above, for instance, the glucose monitoring platform 110 may be configured to store data, such as the glucose measurements 114 associated with a user (e.g., the person 102) and/or users of the user population 108. The glucose monitoring platform 110 may also provide updates and/or additions to the glucose monitoring application 116. Further still, the glucose monitoring platform 110 may train, maintain, and/or deploy algorithms (e.g., machine learning algorithms) to generate or select feedback, such as by using the wealth of data collected from the person 102 and the users of the user population 108. One or more such algorithms may require an amount of computing resources that exceeds the resources of typical, personal computing devices, e.g., mobile phones, laptops, tablet devices, and wearables, to name just a few. Nonetheless, the glucose monitoring platform 110 may include or otherwise have access to the amount of resources needed to operate such algorithms, e.g., cloud storage, server devices, virtualized resources, and so forth. The glucose monitoring platform 110 may provide a variety of resources that the glucose monitoring application 116 leverages in connection with enabling diabetes management feedback to be presented via user interfaces.

In accordance with the described techniques, the glucose prediction system 120 is configured to use the glucose measurements 114 to identify one or more predicted glucose levels and cause output of one or more user interfaces that present the predicted glucose levels. The glucose monitoring application 116 may cause display of the configured user interface 124 via a display device of the computing device 106 or other display device.

As discussed above and below, a variety of prediction-based feedback (e.g., messages) may be selected or generated based on the glucose measurements 114 of the user in accordance with the described techniques. In the context of measuring glucose, e.g., continuously, and obtaining data describing such measurements, consider the following discussion of FIG. 2.

FIG. 2 depicts an example 200 of an implementation of the wearable glucose monitoring device 104 of FIG. 1 in greater detail. In particular, the illustrated example 200 includes a top view and a corresponding side view of the wearable glucose monitoring device 104. It is to be appreciated that the wearable glucose monitoring device 104 may vary in implementation from the following discussion in various ways without departing from the spirit or scope of the described techniques. As noted above, for instance, user interfaces including diabetes management feedback presentation may be configured and displayed (or otherwise output) in connection with other types of devices for glucose monitoring, such as non-wearable devices (e.g., blood glucose meters requiring finger sticks), patches, and so forth.

In this example 200, the wearable glucose monitoring device 104 is illustrated to include a sensor 202 and a sensor module 204. Here, the sensor 202 is depicted in the side view having been inserted subcutaneously into skin 206, e.g., of the person 102. The sensor module 204 is depicted in the top view as a dashed rectangle. The wearable glucose monitoring device 104 also includes a transmitter 208 in the illustrated example 200. Use of the dashed rectangle for the sensor module 204 indicates that it may be housed or otherwise implemented within a housing of the transmitter 208. In this example 200, the wearable glucose monitoring device 104 further includes adhesive pad 210 and attachment mechanism 212.

In operation, the sensor 202, the adhesive pad 210, and the attachment mechanism 212 may be assembled to form an application assembly, where the application assembly is configured to be applied to the skin 206 so that the sensor 202 is subcutaneously inserted as depicted. In such scenarios, the transmitter 208 may be attached to the assembly after application to the skin 206 via the attachment mechanism 212. Alternatively, the transmitter 208 may be incorporated as part of the application assembly, such that the sensor 202, the adhesive pad 210, the attachment mechanism 212, and the transmitter 208 (with the sensor module 204) can all be applied at once to the skin 206. In one or more implementations, this application assembly is applied to the skin 206 using a separate sensor applicator (not shown). Unlike the finger sticks required by conventional blood glucose meters, the user initiated application of the wearable glucose monitoring device 104 is nearly painless and does not require the withdrawal of blood. Moreover, the automatic sensor applicator generally enables the person 102 to embed the sensor 202 subcutaneously into the skin 206 without the assistance of a clinician or healthcare provider.

The application assembly may also be removed by peeling the adhesive pad 210 from the skin 206. It is to be appreciated that the wearable glucose monitoring device 104 and its various components as illustrated are simply one example form factor, and the wearable glucose monitoring device 104 and its components may have different form factors without departing from the spirit or scope of the described techniques.

In operation, the sensor 202 is communicatively coupled to the sensor module 204 via at least one communication channel which can be a wireless connection or a wired connection. Communications from the sensor 202 to the sensor module 204 or from the sensor module 204 to the sensor 202 can be implemented actively or passively and these communications can be continuous (e.g., analog) or discrete (e.g., digital).

The sensor 202 may be a device, a molecule, and/or a chemical which changes or causes a change in response to an event which is at least partially independent of the sensor 202. The sensor module 204 is implemented to receive indications of changes to the sensor 202 or caused by the sensor 202. For example, the sensor 202 can include glucose oxidase which reacts with glucose and oxygen to form hydrogen peroxide that is electrochemically detectable by the sensor module 204 which may include an electrode. In this example, the sensor 202 may be configured as or include a glucose sensor configured to detect analytes in blood or interstitial fluid that are indicative of diabetes management using one or more measurement techniques. In one or more implementations, the sensor 202 may also be configured to detect analytes in the blood or the interstitial fluid that are indicative of other markers, such as lactate levels, which may improve accuracy in generating various diabetes management feedback. Additionally or alternately, the wearable glucose monitoring device 104 may include additional sensors to the sensor 202 to detect those analytes indicative of the other markers.

In another example, the sensor 202 (or an additional sensor of the wearable glucose monitoring device 104 - not shown) can include a first and second electrical conductor and the sensor module 204 can electrically detect changes in electric potential across the first and second electrical conductor of the sensor 202. In this example, the sensor module 204 and the sensor 202 are configured as a thermocouple such that the changes in electric potential correspond to temperature changes. In some examples, the sensor module 204 and the sensor 202 are configured to detect a single analyte, e.g., glucose. In other examples, the sensor module 204 and the sensor 202 are configured to detect multiple analytes, e.g., sodium, potassium, carbon dioxide, and glucose. Alternately or additionally, the wearable glucose monitoring device 104 includes multiple sensors to detect not only one or more analytes (e.g., sodium, potassium, carbon dioxide, glucose, and insulin) but also one or more environmental conditions (e.g., temperature). Thus, the sensor module 204 and the sensor 202 (as well as any additional sensors) may detect the presence of one or more analytes, the absence of one or more analytes, and/or changes in one or more environmental conditions.

In one or more implementations, the sensor module 204 may include a processor and memory (not shown). The sensor module 204, by leveraging the processor, may generate the glucose measurements 114 based on the communications with the sensor 202 that are indicative of the above-discussed changes. Based on these communications from the sensor 202, the sensor module 204 is further configured to generate communicable packages of data that include at least one glucose measurement 114. In one or more implementations, the sensor module 204 may configure those packages to include additional data, including, by way of example and not limitation, a sensor identifier, a sensor status, temperatures that correspond to the glucose measurements 114, measurements of other analytes that correspond to the glucose measurements 114, and so forth. It is to be appreciated that such packets may include a variety of data in addition to at least one glucose measurement 114 without departing from the spirit or scope of the described techniques.

In implementations where the wearable glucose monitoring device 104 is configured for wireless transmission, the transmitter 208 may transmit the glucose measurements 114 wirelessly as a stream of data to a computing device. Alternately or additionally, the sensor module 204 may buffer the glucose measurements 114 (e.g., in memory of the sensor module 204 and/or other physical computer-readable storage media of the wearable glucose monitoring device 104) and cause the transmitter 208 to transmit the buffered glucose measurements 114 later at various intervals, e.g., time intervals (every second, every thirty seconds, every minute, every five minutes, every hour, and so on), storage intervals (when the buffered glucose measurements 114 reach a threshold amount of data or a number of measurements), and so forth.

Having considered an example of an environment and an example of a wearable glucose monitoring device, consider now a discussion of some examples of details of the techniques for diabetes management feedback for improving diabetes management.

### System Architecture

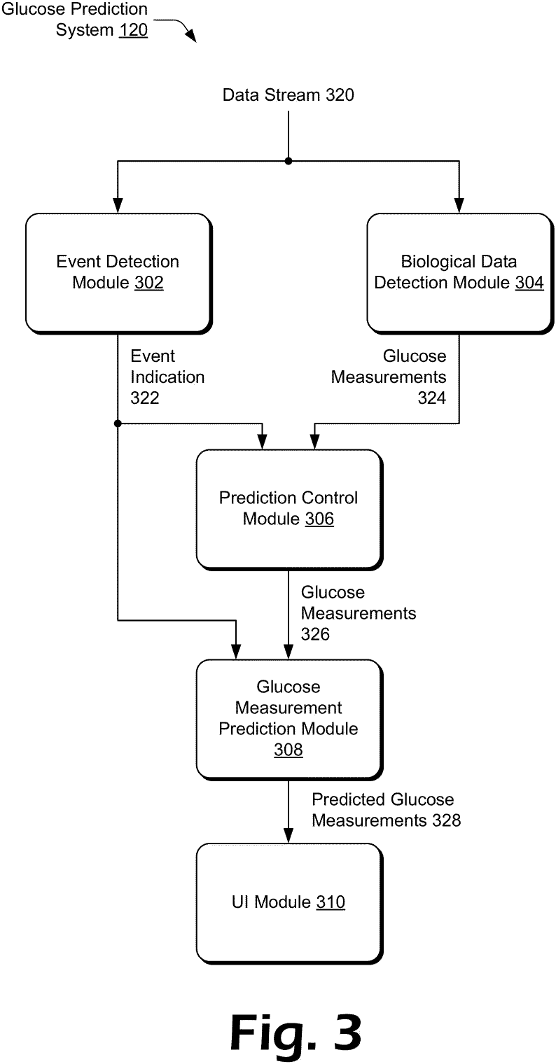

FIG. 3 is an illustration of an example architecture of a glucose prediction system 120. The glucose prediction system 120 includes an event detection module 302, a biological data detection module 304, a prediction control module 306, a glucose measurement prediction module 308, and a UI module 310. Generally, the glucose prediction system 120 analyzes activity data of a user and determines when a period of physical activity occurs. The glucose prediction system 120 predicts what the glucose measurements of the user 102 would have been had the physical activity not occurred, and takes various actions based on the predicted glucose measurements (e.g., provides feedback to the user indicating what their glucose would have been had they not engaged in the physical activity).

The event detection module 302 and biological data detection module 304 each receive a data stream 320. The data in the data stream 320 can be received from various different sources, such as the wearable glucose monitoring device 104, one or more sensors of the computing device 106, another sensor or device worn by the user 102, user inputs (e.g., specifying times when particular activities occurred or actions were taken by the user, specifying measurements received from various sensors), a local or remote database (e.g., accessed via the network 112), and so forth. The data in the data stream 320 can include data received at regular intervals (e.g., approximately every 5 minutes), single occurrence data (e.g., data input via a user interface, such as data describing a meal eaten at a particular time), and so forth. In one or more implementations, the data stream 320 includes glucose measurements 114 and timestamps indicating when each of the glucose measurements 114 was taken (e.g., by wearable glucose monitoring device 104) or received (e.g., by glucose monitoring application 116). The timestamp may be provided, for example, by the wearable glucose monitoring device 104 or the glucose monitoring application 116. Additionally or alternatively, the data stream 320 includes any of a variety of other data that indicates events or conditions that may affect glucose of the user 102 (e.g., glucose levels of the user 102), such as activities the user 102 engages in, behaviors of the user 102, reactions of the user 102, medical conditions of the user 102, biological data of the user 102, and so forth.

In one or more implementations, the data stream 320 includes physical activity data, such as a number of steps walked over a particular range of time (e.g., every 10 seconds, every minute), heart rate over a particular range of time (e.g., at regular or irregular intervals, such as every 15 seconds) with timestamps, speed of movement with timestamp (e.g., at regular or irregular intervals, such as every 15 seconds), raw or filtered accelerometer data, and so forth. Physical activity data can be received from various sources, such as wearable glucose monitoring device 104, an activity tracking application running on computing device 106, an activity or fitness tracker worn by the user 102, and so forth.

Additionally or alternatively, data stream 320 includes meal data. E.g., this meal data can include timestamps of when the user 102 ate and what foods were consumed, timestamps of when particular types or classes of foods were consumed (e.g., vegetables, grain, meat, sweets, soda), amounts of food consumed, and so forth.

Additionally or alternatively, data stream 320 includes sleep data, such as data indicating minutes of the day when the user was sleeping. Sleep data can also include data regarding sleeping patterns of the user. E.g., data stream 320 can include data indicating times when the user is sleeping, the sleep state (e.g., Stage 1, Stage 2, Stage 3, or rapid eye movement (REM) sleep) of the user at particular times, and so forth. Sleep data can be received from various sources, such as wearable glucose monitoring device 104, a sleep tracking application running on computing device 106, an activity or fitness tracker worn by the user 102, and so forth.

Additionally or alternatively, data stream 320 includes medication data. E.g., this medication data can include timestamps of when user 102 took medicine and what medicine was taken (which can be used to determine whether the user 102 is taking his or her medicine at the prescribed times or intervals), indications of changes in medicines (e.g., changes in types or dosages of medicines taken), and so forth.

Additionally or alternatively, data stream 320 includes data that reflects stress management, such as heart rate variability (HRV), skin conductivity and temperature, respiration rate measurements, data from an electroencephalogram (EEG), cortisol in biofluids, volatile organic components (VOCs) emitted from the skin, and so forth.

Additionally or alternatively, data stream 320 includes data regarding user engagement with glucose monitoring application 116. E.g., this application engagement data can include timestamps of when the user 102 viewed the application as well as what screens or portions of the UI were viewed, timestamps of when the user 102 provided input to (or otherwise interacted with) the application 116 as well as what that input was, timestamps of when the user viewed or acknowledged feedback provided by the application 116, and so forth.

Additionally or alternatively, data stream 320 include data that relates to user interactions with the computing device 106, with display of the computing device 106, or with other system components that indicate level of engagement with diabetes management. Examples of such data include the number of times applications (e.g., glucose monitoring application) is opened, the time spent reviewing glucose data or previous feedback or educational materials, the frequency of interactions with coaches or clinicians, and so forth.

Additionally or alternatively, data stream 320 includes data regarding user engagement with others of user population 108, such as via glucose monitoring platform 110. E.g., this other-user engagement data can include timestamps of when the user 102 communicated with another user as well as who that other user was, descriptions of what information was communicated with another user, and so forth.

The event detection module 302 receives data stream 320 and identifies events or conditions in the data stream 320 that may affect glucose levels of the user. These events or conditions can be any event or condition indicated by the data in the data stream 320, such as physical activity, sleep, meals consumed, medication taken, and so forth. The event detection module 302 outputs an event indication 322 that identifies these events or conditions, such as an indication of a bout of physical activity by the user 102, an indication of a time the user 102 was sleeping, an indication of meals consumed by the user 102, an indication of medication taken by the user 102, and so forth.

In one or more implementations, the event detection module 302 receives physical activity data in data stream 320 and identifies bouts of physical activity by the user 102. Physical activity refers to any bodily movement produced by skeletal muscle that results in energy expenditure above resting (basal) levels. The event detection module 302 identifies a bout of physical activity, which is an amount of time during which the energy expenditure by the user is at least a threshold amount above resting levels. The event detection module 302 identifies bouts of physical activity in any of a variety of different manners. In one or more implementations, the event detection module 302 identifies a bout of physical activity based on a number of steps taken. For example, a bout of physical activity is user 102 taking at least a threshold number of steps (e.g., 60) per minute for at least a threshold amount of time (e.g., 5 minutes) and without dropping below the threshold number of steps (e.g., 60) for at least a consecutive amount of time (e.g., 5 minutes). The bout ends when the user 102 drops below the threshold number of steps (e.g., 60) for at least the consecutive number of minutes (e.g., 5 minutes). Allowing the number of steps to drop below the threshold number of steps for less than the consecutive amount of time allows a single bout of physical activity to be identified even though the user takes small resting breaks during the physical activity. These thresholds (e.g., threshold amount of time or threshold number of steps) are optionally adjusted or modified based on various characteristics of the user such as their age, fitness level, prevalence of co-morbidities that may affect walking gate speed, and so forth. E.g., Older individuals may require more conservative thresholds to reach the same intensity as a younger individual with higher thresholds.

Additionally or alternatively, the event detection module 302 identifies a bout of physical activity based on any of various heart-rate based intensity values. One such heart-rate based intensity value is a percent heart rate reserve value for user 102. The percent heart rate reserve value indicates how close the user is to their estimated max heart rate. For example, the percent heart rate reserve (%HHR) value for a user at a current time can be identified as:

$$\% H H R = \frac{H {R}_{e x} - H {R}_{r e s t}}{H R R} ∗ 100$$

where HRₑₓ refers to the heart rate of the user at the current time, HRᵣₑₛₜ refers to the resting heart rate of the user, and HRR refers to the heart rate reserve of the user 102, which is determined as HRR = HRₘₐₓ - HRᵣₑₛₜ, where HRmₐₓ refers to the max heart rate of the user.

The current heart rate of a user is obtained in various manners, such as from an activity monitor worn by the user. The resting heart rate of the user is obtained in various manners, such as from an activity monitor worn by the user, input from the user via a UI (e.g., of computing device 106), and so forth. The max heart rate of the user is obtained in various manners, such as from a VO₂ max test, estimated from various formulas, and so forth.

The event detection module 302 uses the percent heart rate reserve value in various manners to determine a bout of physical activity for the user 102. For example, a bout of physical activity is the percent heart rate reserve value for the user 102 exceeding a threshold amount (e.g., 40%) for at least a threshold amount of time (e.g., 3 minutes) and without dropping below the threshold amount (e.g., 40%) for at least a consecutive amount of time (e.g., 3 minutes). The bout ends when the user 102 drops below the threshold amount (e.g., 40%) for at least the consecutive amount of time (e.g., 3 minutes). Allowing the percent heart rate reserve to drop below the threshold amount for less than the consecutive amount of time allows a single bout of physical activity to be identified even though the user takes small resting breaks during the physical activity.

Another such heart-rate based intensity value is a percent of max heart rate. The max heart rate of the user is obtained in various manners as discussed above. The event detection module 302 uses the percent of max heart rate in various manners to determine a bout of physical activity for the user 102. For example, a bout of physical activity is the max heart rate for the user 102 exceeding a threshold amount (e.g., 60%) for at least a threshold amount of time (e.g., 3 minutes) and without dropping below the threshold amount (e.g., 60%) for at least a consecutive amount of time (e.g., 3 minutes). The bout ends when the user 102 drops below the threshold amount (e.g., 60%) for at least the consecutive amount of time (e.g., 3 minutes). Allowing the max heart rate to drop below the threshold amount for less than the consecutive amount of time allows a single bout of physical activity to be identified even though the user takes small resting breaks during the physical activity.

Additionally or alternatively, the event detection module 302 identifies a bout of physical activity based on Metabolic Equivalents (METs) for user 102. METs are an estimate of the amount of energy used relative to the user sitting at rest, and one MET is the amount of oxygen consumed by the user while sitting at rest. The METs expended by a user at any a current time is obtained in various manners, such as from an activity monitor worn by the user.

The event detection module 302 uses METs in various manners to determine a bout of physical activity for the user 102. For example, a bout of physical activity is the number of METs for the user 102 exceeding a threshold amount (e.g., 2 METs) for at least a threshold amount of time (e.g., 5 minutes) without dropping below the threshold amount (e.g., 2 METs) for at least a consecutive amount of time (e.g., 5 minutes). The bout ends when the user 102 drops below the threshold amount (e.g., 2 METs) for at least the consecutive number of minutes (e.g., 5 minutes). Allowing the METs to drop below the threshold amount for less than the consecutive amount of time allows a single bout of physical activity to be identified even though the user takes small resting breaks during the physical activity.

The event detection module 302 may also use multiple different techniques concurrently to identify a bout of physical activity. In such situations, the threshold amounts or values may vary from when a single technique is used. For example, a bout of physical activity is the percent heart rate reserve value for the user 102 exceeding a threshold amount (e.g., 45%) and the user 102 taking at least a threshold number of steps (e.g., 40) per minute for at least a threshold amount of time (e.g., 5 minutes). The bout continues for as long as the user 102 does not drop below the threshold amount (e.g., 45% heart rate reserve and 40 steps per minute) for at least a consecutive amount of time (e.g., 5 minutes). The bout ends when the user 102 drops below the threshold amount (45% heart rate reserve and 40 steps per minute) for at least the consecutive amount of time (e.g., 5 minutes ). This combination allows, for example, a smaller number of steps to be identified as a bout of physical activity if the user’s heart rate is high enough.

Additionally or alternatively, bouts of physical activity can be identified in various other manners. For example, user input (e.g., voice input, gesture, selection of a button on computing device 106) can be received indicating the beginning and the ending of a bout of physical activity. By way of another example, a bout of physical activity may begin when a heart rate monitor (e.g., worn by the user) is turned on and end when the heart rate monitor is turned off. By way of another example, a bout of physical activity may begin when a heart rate monitor (e.g., worn by the user) is detected by an exercise machine (e.g., a treadmill or other exercise machine) such as via Bluetooth or ANT communications, and ends when the heart rate monitor is no longer detected by the machine. The exercise machine can communicate, for example, the beginning and ending of the bout of physical activity to the computing device 106.

The event detection module 302 outputs an event indication 322 to the prediction control module 306 as well as to the glucose measurement prediction module 308 for each identified bout of physical activity. Each event indication 322 indicates a duration of time during which the bout of physical activity occurred. This time can be, for example, the beginning and ending times of the bout of physical activity.

The biological data detection module 304 receives data stream 320 and identifies glucose measurements in the data stream 320. These identified glucose measurements are provided to the prediction control module 306 as glucose measurements 324. Additionally or alternatively, the biological data detection module 304 detects any of a variety of other data included in the data stream 320, such as heart rate data, HRV data, respiration rate data, and so forth that may affect glucose of the user 102 and provides that detected data to prediction control module 306. Additionally or alternatively, the biological data detection module 304 may detect other types of information from data stream 320 (e.g. from a database on premises or in the cloud via network 112) based off what information the glucose prediction system 120 has about the user and cohorts (e.g., other users in the user population 108) having similar characteristics as the user to aid in predicting glucose measurements. For example, if biological data detection module 304 has not detected information regarding the fitness level of the user (e.g., in situations in which the fitness level of the user is used by the glucose measurement prediction module 308 in generating predicted glucose measurements), the biological data detection module 304 detects or retrieves demographic information of the individual and estimates their fitness level based on the fitness level of their most similar cohort. The biological data detection module 304 can provide any of this data or information to the glucose measurement prediction module 308 for generation of the predicted glucose measurements.

The prediction control module 306 identifies, for a bout of physical activity identified in an event indication 322, an amount of time immediately preceding the bout of physical activity. This amount of time can be, for example, 30-40 minutes. The prediction control module 306 identifies which glucose measurements 324 correspond to the amount of time immediately preceding the bout of physical activity (e.g., have timestamps within the 30-40 minutes immediately preceding the bout of physical activity) and provides the glucose measurements to the glucose measurement prediction module 308 as glucose measurements 326.

The glucose measurement prediction module 308 receives the event indication 322 and the glucose measurements 326 and predicts an impact of the physical activity bout on glucose of the user. The glucose measurement prediction module 308 generates this prediction by generating, based on the glucose measurements 326 (and optionally other physiological or demographic data), one or more predicted glucose measurements that the user would have had if, during the time the user was performing the bout of physical activity (as indicated by event indication 322), the user had not performed the bout of physical activity. The predicted glucose measurements are output as predicted glucose measurements 328.

In one or more implementations, the glucose measurement prediction module 308 includes a machine learning system that generates the predicted glucose measurements. Machine learning systems refer to a computer representation that can be tuned (e.g., trained) based on inputs to approximate unknown functions. In particular, machine learning systems can include a system that utilizes algorithms to learn from, and make predictions on, known data by analyzing the known data to learn to generate outputs that reflect patterns and attributes of the known data. For instance, a machine learning system can include statistical time series forecasting models such as single order auto regressive models and second order auto regressive models, decision trees, support vector machines, linear regression, logistic regression, Bayesian networks, random forest learning, dimensionality reduction algorithms, boosting algorithms, artificial neural networks, deep learning, and so forth.

The machine learning system is trained, for example, by using training data that is sets of multiple glucose measurements for the user. These are, for example, sets of multiple consecutive glucose measurements over an amount of time (the same amount of time for which glucose measurements immediately preceding a bout of physical activity are identified by the prediction control module 306, such as 30-40 minutes). The training data can be selected (e.g., randomly or pseudorandomly) based on glucose measurements received for a user over various days, weeks, months, and so forth. The training data includes glucose measurements for amounts of time that do not include bouts of physical activity. This allows the machine learning system to be trained to predict glucose measurements that occur in the absence of physical activity.

Known labels are associated with the sets of multiple data indicating what the subsequent glucose measurements were (e.g., the glucose measurements that occur immediately after those in the training data). The machine learning system is trained by updating weights or values of layers or coefficients in the machine learning system to minimize the loss between glucose measurements generated by the machine learning system for the training data and the corresponding known labels for the training data. Various different loss functions can be used in training the machine learning system, such as cross entropy loss, mean squared error loss, and so forth.

Additionally or alternatively, the machine learning system is trained to generate the predicted glucose measurements based on any of a variety of other data in the data stream 320 or detected by the biological data detection module 304. In such situations, the training data includes sets of the data for the user, such as sets of multiple data measured over an amount of time. For example, the machine learning system can be trained to generate the predicted glucose measurements based on any combination of physiological parameters (e.g., raw heart rate data, relative heart rate-based intensity measures, blood pressure measures, and so forth), demographic information (e.g., age, gender, and so forth), clinical information (medication stack data, prevalence of comorbidities data, fitness level data, etc.), and so forth.

The machine learning system is trained to generate multiple glucose measurements that occur after the training data (e.g., immediately after the training data). The number of glucose measurements the machine learning system is trained to generate can be determined in a variety of different manners, such as determining an average duration for a bout of physical activity for the user based on previous bouts of physical activity for the user, receiving user input specifying the typical duration of a bout of physical activity for the user, and so forth. In one or more implementations, the machine learning system is trained to generate a number of glucose measurements following the training data that would typically be measured during a bout of physical activity (e.g., during the average duration or typical duration for a bout of physical activity). For example, assuming glucose measurements are obtained every 5 minutes and the typical duration of a bout of physical activity is 30 minutes, the machine learning system would be trained to generate a predicted glucose measurement after 5 minutes, after 10 minutes, after 15 minutes, after 20 minutes, after 25 minutes, and after 30 minutes. Additionally or alternatively, the machine learning system can be trained on data that is not in the immediate vicinity of the prediction time point (e.g., there could be a gap between the training period and the prediction time point).

Additionally or alternatively, the machine learning system is trained to generate a number of glucose measurements following the training data that would typically be measured during a bout of physical activity (e.g., during the average duration or typical duration for a bout of physical activity) and extending beyond the bout of physical activity by some duration of time (e.g., 15 or 20 minutes). For example, assume glucose measurements are obtained every 5 minutes and the typical duration of a bout of physical activity is 30 minutes, the machine learning system would be trained to generate a predicted glucose measurement after 5 minutes, after 10 minutes, after 15 minutes, after 20 minutes, after 25 minutes, after 30 minutes, after 35 minutes, after 40 minutes, and after 45 minutes.

In one or more implementations, the machine learning system generates a confidence level for each predicted glucose measurement. In such situations, the glucose prediction system 120 can take various actions based on the confidence levels for the predicted glucose measurements. For example, the glucose prediction system 120 may notify the user of predicted glucose measurements (as discussed in more detail below) only in situations where the confidence level of the predicted glucose measurements exceeds a threshold value (e.g., 75%). By way of another example, the glucose prediction system 120 may only notify the user of predicted glucose measurements for as long as the confidence level for the glucose measurements exceeds a threshold value (e.g., 75%) -after the confidence level no longer exceeds the threshold value the glucose prediction system 120 no longer notifies the user of the predicted glucose measurements regardless of whether the user is still engaged in a bout of physical activity.

In one or more implementations, the machine learning system generates a prediction interval for each predicted glucose measurements. For example, for the predicted glucose measurements after 10 minutes, a prediction interval or range is generated, such as a range of predicted glucose measurements having a confidence level that exceeds a threshold value (e.g., 75%). In such implementations, the glucose prediction system 120 may only notify the user of predicted glucose measurements if the actual glucose measurement of the user at the time of the bout of physical activity is outside of the prediction interval or range, or is beyond some threshold value (e.g., 250 mg/dL). Accordingly, the user need not be notified of situations where there is not a meaningful difference between their actual glucose measurement and the predicted glucose measurement had they engaged in a bout of physical activity).

Additionally or alternatively, the glucose measurement prediction module 308 can use any of various other models to generate the predicted glucose measurements 328. For example, the glucose measurement prediction module 308 can use physiological (pharmo-kinetics) or phenomenological models. E.g., glucose uptake can be modeled using ordinary differential equations that have parameters such as glucose uptake and exercise intensity.

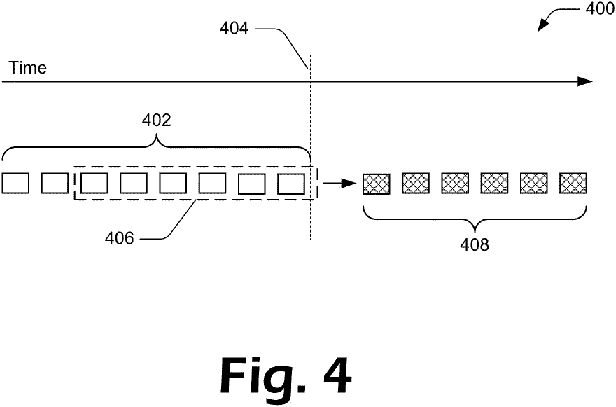

FIG. 4 illustrates an example 400 of generating predicted glucose measurements. In the example 400, multiple (eight) glucose measurements 402 are illustrated (e.g., received as glucose measurements 324). A time 404 is illustrated that corresponds to the beginning of a bout of physical activity for the user (e.g., as indicated by the event indication 322). The glucose measurements 406 are a subset of the glucose measurements 402 and are the glucose measurements immediately preceding the time 404. The glucose measurements 406 are used by the glucose measurement prediction module 308 to generate predicted glucose measurements 408 that occur immediately after the glucose measurements 402. The predicted glucose measurements 408 are generated for the duration of the bout of physical activity that began at the time 404. Additionally or alternatively, the predicted glucose measurements 408 may be generated for other durations of time, such as an amount of time (e.g., 15 or 20 minutes) extending beyond the bout of physical activity. This allows more meaningful glycemic impact feedback to be provided to the user 102. For example, the bout of physical activity is only 8 minutes in duration, extending the predicted glucose measurements 408 by 15 or 20 minutes allows more accurate feedback to be provided to the user accounting for the time the user’s body takes to react to the physical activity and make a meaningful change in the user’s glucose measurements.

By way of another example, the predicted glucose measurements 408 may be generated for different durations of time based on the intensity of the physical activity. For example, the higher the intensity of the physical activity, the longer the duration of time that the predicted glucose measurements 408 are generated for.

Returning to FIG. 3, the training data used to train the machine learning system includes glucose measurements for the particular user 102. Accordingly, the machine learning system of the glucose measurement prediction module 308 is trained or customized to the individual user 102, accounting for that individual user’s body and glucose.

Although customized to the individual user 102, the machine learning system of the glucose measurement prediction module 308 can optionally be re-trained over time in response to various events that may alter glucose management for the user. For example, the machine learning system can be re-trained using new training data after some period of time (e.g., 6 months or 1 year) to account for changes in the user’s body. By way of another example, the machine learning system can be retrained using new training data obtained after a change in medication for the user.

The UI module 310 receives the predicted glucose measurements 328 and causes the predicted glucose measurements 328 to be displayed or otherwise presented (e.g., at computing device 106). This display or other presentation can take various forms, such as a static text display, graphic or video display, audio presentation, combinations thereof, and so forth. Additionally or alternatively the predicted glucose measurements 328 are communicated to another user or system, such as to a health care provider or clinician. The glucose measurement prediction module 308 optionally incorporates the predicted glucose measurements 328 into a message or feedback to the user, such as a congratulatory message identifying the improvement in glucose (as indicated by the predicted glucose measurements 328) over what the user’s glucose measurements would have been without the bout of physical activity.

The UI module 310 can display or otherwise present predicted glucose measurements 328 at any of a variety of times. In one or more implementations, the UI module 310 displays or otherwise presents predicted glucose measurements 328 at the ending of a bout of physical activity. Additionally or alternatively, the UI module 310 displays or otherwise presents predicted glucose measurements 328 at other times, such as in response to a user request for the predicted glucose measurements 328, at particular time intervals (e.g., every evening or every morning), in response to a positive and meaningful change in glucose levels or dynamics (e.g., the user’s glucose level dropped below a threshold amount or dropped by a threshold amount), and so forth.

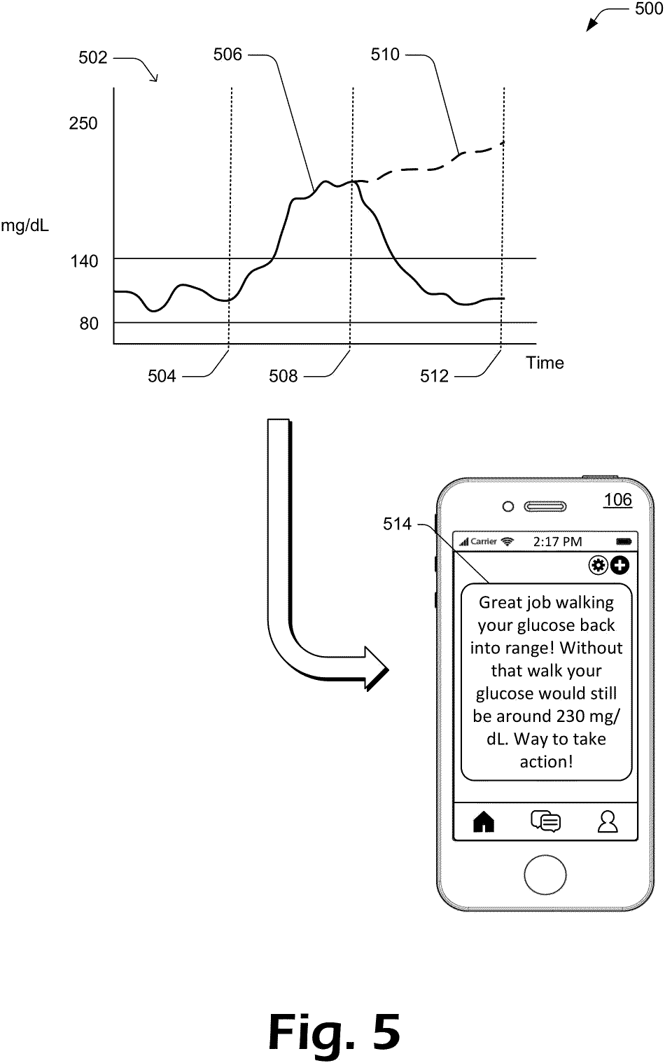

FIG. 5 illustrates an example 500 of providing predicted glucose measurements. The example 500 includes a graph 502 plotting glucose measurements in mg/dL along the vertical axis against time along the horizontal axis. In the example 500, assume that the user eats a meal at time 504. The user’s glucose measurements illustrated by the solid line 506 increase after eating the meal. Further assume that the user begins a bout of physical activity at time 508. As a result of the physical activity, the user’s glucose measurements begin decreasing as shown. The glucose prediction system 120 generates predicted glucose measurements illustrated by the dashed line 510 beginning at time 508 (the beginning of the bout of physical activity) through time 512 (e.g., the ending of the bout of physical activity). The glucose prediction system 120 provides feedback 514 for display on the computing device 106 providing an indication of the predicted glucose measurements and the actual glucose measurements for the user. As illustrated, the feedback 514 indicates the result of the bout of physical activity on the user’s glucose and indicates how much better the user’s glucose is than if he had not performed the bout of physical activity.

Returning to FIG. 3, the glucose measurement prediction module 308 is discussed as providing the predicted glucose measurements 328 to the UI module 310. The glucose prediction system 120 optionally takes additional actions based on the predicted glucose measurements 328. In one or more implementations, these actions include notifying the glucose monitoring application 116 or the wearable glucose monitoring device 104 that the frequency with which glucose measurements 114 are produced can be reduced. For example, if the glucose prediction system 120 identifies a bout of physical activity, and the predicted glucose measurements 328 for previous bouts of physical activity indicate an improvement in glucose measurements over the user 102 not engaging in the bout of physical activity, the glucose prediction system 120 can notify the glucose monitoring application 116 or wearable glucose monitoring device 104 that the frequency with which glucose measurements 114 are produced during a bout of physical activity can be reduced (e.g., from every 5 minutes to every 10 minutes), reducing the power expended to produce glucose measurements 114.

The discussions of the glucose prediction system 120 also include generating predicted glucose measurements 328 in response to detecting bouts of physical activity. Additionally or alternatively, the glucose prediction system 120 generates predicted glucose measurements 328 based on bouts of physical activity relative to other events, conditions, biological data, and so forth (e.g., based on any data in the data stream 320). For example, the glucose prediction system 120 may generate predicted glucose measurements 328 in response to physical activity occurring within a threshold amount of time (e.g., 30 minutes) of the user eating or drinking.

Furthermore, the discussions of the glucose prediction system 120 include predicting glucose measurements during bouts of physical activity. In one or more implementations, the glucose prediction system 120 differentiates between or among multiple different types of physical activity. For example, the event detection module 302 may detect different types of physical activity, such as slow walking (e.g., at 60-79 steps per minute), medium walking (at 80-99 steps per minute), brisk walking (e.g., at 100-119 steps per minute), resistance training, and so forth. The glucose measurement prediction module 308 can include a machine learning system trained using training data obtained during one of these types of physical activity, and can predict glucose measurements for the user for one of type of physical activity when a bout of another type of physical activity was performed by the user. For example, a machine learning system may be trained using training data obtained during bouts of slow walking. Subsequently, when the user engages in a bout of brisk walking, the glucose measurement prediction module 308 can generate predicted glucose measurements 328 indicating glucose if the user had instead engaged in a bout of slow walking. These predicted glucose measurements 328 can be displayed or otherwise provided to the user, notifying the user of the improved glucose measurements resulting from brisk walking over slow walking.

Additionally or alternatively, in one or more implementations the glucose prediction system 120 predicts glucose measurements during times when the user is not engaged in a bout of physical activity. Such predicted glucose measurements can be generated analogous to the discussion herein regarding predicting glucose measurements during bouts of physical activity, except that the glucose measurement prediction module 308 includes a machine learning system trained to generate predicted glucose measurements during a bout of physical activity. This allows the glucose prediction system 120 to provide feedback to the user or other person or system indicating what the user’s predicted glucose measurements would be if the user had in fact engaged in a bout of physical activity.

Additionally or alternatively, the glucose prediction system 120 can predict glucose measurements based on any data included in the data stream 320, such as data that indicates events or conditions that may affect glucose of the user 102. Such predicted glucose measurements can be generated analogous to the discussion herein regarding predicting glucose measurements during bouts of physical activity. This allows the glucose prediction system 120 to predict glucose measurements for other bouts or durations of time during which other activities or biological reactions are occurring.

By way of example, the data stream 320 may include meal data. Accordingly, the glucose measurement prediction module 308 can include a machine learning system trained using training data obtained over amounts of time when the user was not eating or drinking (and optionally what type of food or drink was being consumed by the user). This allows the glucose measurement prediction module 308 to predict, for a duration of time during or after eating or drinking, glucose measurements for the user if the user had not consumed any food or drink (or had consumed a different type of food or drink). The differences between the actual glucose measurements and the predicted glucose measurements for the user if the user had not consumed any food or drink (or had consumed a different type of food or drink) can be displayed or otherwise provided to the user or other person or system.

By way of another example, the data stream 320 may include sleep data. Accordingly, the glucose measurement prediction module 308 can include a machine learning system trained using training data obtained over amounts of time when the user was not sleeping (or was in a particular sleep state). This allows the glucose measurement prediction module 308 to predict, for a duration of time during or after sleeping, glucose measurements for the user if the user had not slept (or had slept in a different sleep state or for a different duration of time). The differences between the actual glucose measurements and the predicted glucose measurements for the user if the user had not slept (or had slept in a different sleep state or for a different duration of time) can be displayed or otherwise provided to the user or other person or system.

By way of another example, the data stream 320 may include medication data. Accordingly, the glucose measurement prediction module 308 can include a machine learning system trained using training data obtained over amounts of time when the user did take medication (and optionally what type or dose of medication was taken by the user). This allows the glucose measurement prediction module 308 to predict, for a duration of time during or after taking medication, glucose measurements for the user if the user had not taken the medication (or had taken a different type or dose of medication). The differences between the actual glucose measurements and the predicted glucose measurements for the user if the user had not taken the medication (or had taken a different type or dose of medication) can be displayed or otherwise provided to the user or other person or system.

By way of another example, the data stream 320 may include data that reflects stress management. Accordingly, the glucose measurement prediction module 308 can include a machine learning system trained using training data obtained over amounts of time when the user was not stressed (or highly stressed). The user being stressed or highly stressed can be determined in various manners, such as various biological data (e.g., HRV, skin conductivity and temperature, respiration rate, EEG data, cortisol in biofluids, VOCs emitted from the skin) exceeding one or more threshold values, received user feedback on how stressed they are (e.g., via the glucose monitoring application 116 or other mobile application or desktop user interface), such as a rating on a 1-10 stress scale, and so forth. This allows the glucose measurement prediction module 308 to predict, for a duration of time when the user is stressed (or highly stressed), glucose measurements for the user if the user were not stressed (or was not highly stressed). The differences between the actual glucose measurements and the predicted glucose measurements for the user if the user were not stressed (or not highly stressed) can be displayed or otherwise provided to the user or other person or system.

By way of another example, the data stream 320 may include data regarding user engagement with glucose monitoring application 116. Accordingly, the glucose measurement prediction module 308 can include a machine learning system trained using training data obtained over amounts of time when the user was not engaged with the glucose monitoring application 116 or was engaged with the glucose monitoring application 116 in a particular manner (e.g., what screens were viewed or what data was input). This allows the glucose measurement prediction module 308 to predict, for a duration of time during or after engaging with the glucose monitoring application 116 (or engaging with the glucose monitoring application 116 in a particular manner), glucose measurements for the user if the user had not engaged with the glucose monitoring application 116 (or had engaged with the glucose monitoring application 116 in a different manner). The differences between the actual glucose measurements and the predicted glucose measurements for the user if the user had not engaged with the glucose monitoring application 116 (or had engaged with the glucose monitoring application 116 in a different manner) can be displayed or otherwise provided to the user or other person or system.

By way of another example, the data stream 320 may include user interaction data that relates to user interactions with the computing device 106, with display of the computing device 106, or with other system components that indicate level of engagement with diabetes management. Accordingly, the glucose measurement prediction module 308 can include a machine learning system trained using training data obtained over amounts of time when the user was interacting with the computing device 106, the display, or other system components (or optionally of what type of interaction the user had). This allows the glucose measurement prediction module 308 to predict, for a duration of time during or after interacting with the computing device 106, the display, or other system components, glucose measurements for the user if the user had interacted with the computing device 106, the display, or other system (or had interacted with a different one of the computing device 106, the display, or other system). The differences between the actual glucose measurements and the predicted glucose measurements for the user if the user had interacted with the computing device 106, the display, or other system (or had interacted with a different one of the computing device 106, the display, or other system) can be displayed or otherwise provided to the user or other person or system.

By way of another example, the data stream 320 may include data regarding user engagement with others of user population 108. Accordingly, the glucose measurement prediction module 308 can include a machine learning system trained using training data obtained over amounts of time when the user was not engaged with other users of user population 108 (or optionally which other users of the user population 108 the user was engaged with). This allows the glucose measurement prediction module 308 to predict, for a duration of time during or after engaging with other users of user population 108, glucose measurements for the user if the user had not engaged with other users of user population 108 (or had engaged with different users of the user population 108). The differences between the actual glucose measurements and the predicted glucose measurements for the user if the user had not engaged with other users of user population 108 (or had engaged with different users of the user population 108) can be displayed or otherwise provided to the user or other person or system.

Various different machine learning systems are discussed herein (e.g., for different types of data, different types of physical activity, and so forth). It should be noted that the glucose prediction system 120 can include a single one of these machine learning systems or any combination of the machine learning systems discussed herein. Accordingly, any of the predicted glucose measurements discussed herein can be generated concurrently with any other of the predicted glucose measurements.

The glucose measurement prediction module 308 is discussed as including a machine learning system trained based on glucose measurements of the particular user 102. Additionally or alternatively, users are separated into different populations that have one or more similar characteristics. The user 102 is part of one of these different populations and the machine learning systems of the glucose measurement prediction module 308 are trained using training data obtained from other users that are in the same population as the user 102 (e.g., and excluding any data obtained from users that are not in the same population as the user 102).

The populations can be defined in any of a variety of different manners. In one or more embodiments, the populations are defined by diabetes diagnosis (e.g., the user does not have diabetes, the user has Type 1 diabetes, or the user has Type 2 non-insulin-dependent diabetes). Additionally or alternatively, the populations are defined in different manners, for example age-based populations. E.g., populations are based on whether the user is an adult or a child (e.g., older than 18 or younger than 18), based on an age bracket the user is in (e.g., 0-5 years old, 5-10 years old, 10-20 years old, 20-30 years old, etc.), and so forth. By way of another example, populations can be defined based on additional medical conditions a user may have, such as hypertension, obesity, cardiovascular disease, neuropathy, nephropathy, retinopathy, Alzheimer’s, depression, and so forth. By way of another example, populations can be defined based on user habits or activities, such as exercise or other physical activities, sleep patterns, time spent working versus at leisure, and so forth. By way of another example, populations can be defined based on the manner in which glucose measurements 114 are obtained or the equipment used to obtain glucose measurements 114, such as whether glucose measurements 114 are obtained via CGM, a brand of wearable glucose monitoring device 104, a frequency with which glucose measurements 114 are obtained, and so forth.

By way of another example, populations can be defined based on past glucose measurements 114 for users, such as by grouping users by clustering based on past glucose measurements 114. Examples of such clusters include users with high glycemic variability, users with frequent hypoglycemia, users with frequent hyperglycemia, and so forth. By way of another example, users can be grouped by clustering by using the past activity data of the users (e.g., step counts, energy expenditure, exercise minutes, sleep hours, and so forth obtained from activity trackers worn by the users). Examples of such clusters include users with high average steps per day, users with low average energy expenditure per day, users with low average number of sleep hours, and so forth.

Having discussed exemplary details of the techniques for glycemic impact prediction for improving diabetes management, consider now some examples of procedures to illustrate additional aspects of the techniques.

### Example Procedures

This section describes examples of procedures for implementing glycemic impact prediction for improving diabetes management. Aspects of the procedures may be implemented in hardware, firmware, or software, or a combination thereof. The procedures are shown as a set of blocks that specify operations performed by one or more devices and are not necessarily limited to the orders shown for performing the operations by the respective blocks.

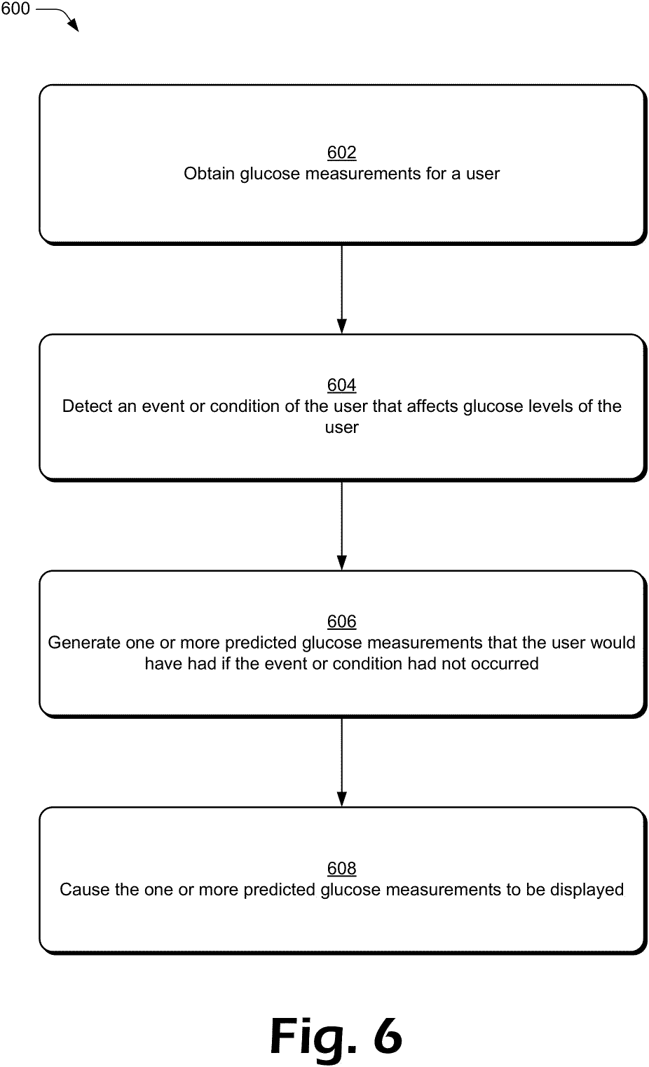

FIG. 6 depicts a procedure 600 in an example of implementing glycemic impact prediction for improving diabetes management. Procedure 600 is performed, for example, by a diabetes management feedback generation system, such as the glucose prediction system 120.

Glucose measurements for a user are obtained (block 602). These glucose measurements are obtained from a glucose sensor of, for example, a continuous glucose level monitoring system with the glucose sensor being inserted at an insertion site of the user.

An event or condition of the user that affects glucose levels of the user is detected (block 604). Any of a variety of events or conditions can be detected, such as bouts of physical activity, meals consumed, sleep, and so forth.

One or more predicted glucose measurements are generated (block 606). The one or more predicted glucose measurements are glucose measurements that the user would have had if the event or condition had not occurred. These predicted glucose measurements are a prediction of the impact of the event or condition on glucose of the user.

The predicted glucose measurements are caused to be displayed (block 608) or otherwise presented. Additionally or alternatively, the predicted glucose measurements can be communicated to or otherwise presented to a clinician, pharmacist, or other health care provider.

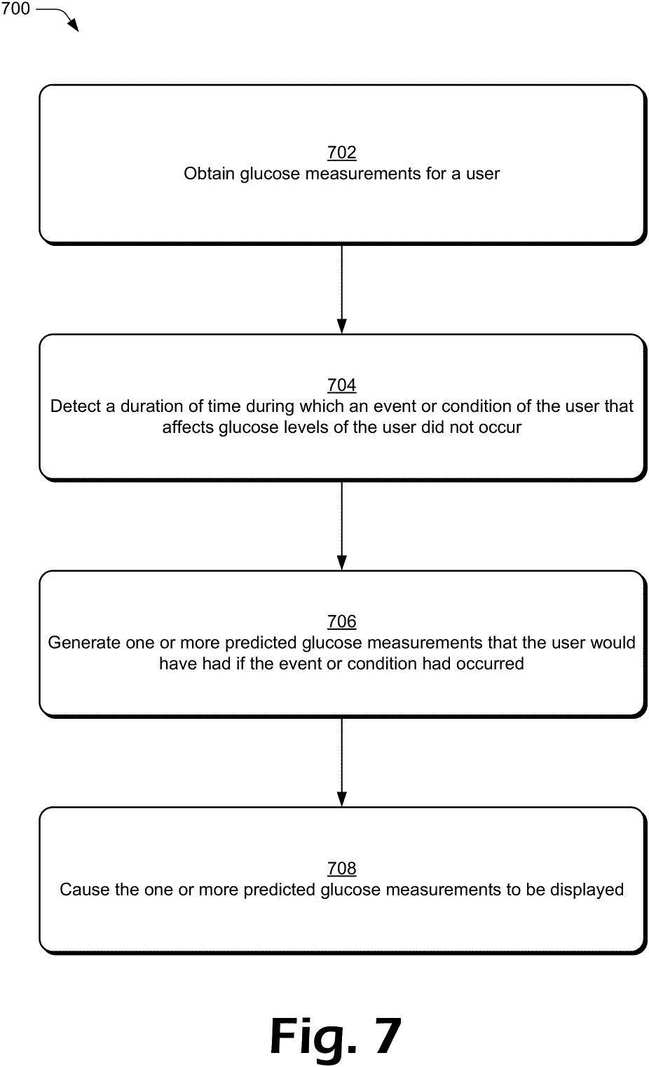

FIG. 7 depicts a procedure 700 in an example of implementing glycemic impact prediction for improving diabetes management. Procedure 700 is performed, for example, by a diabetes management feedback generation system, such as the glucose prediction system 120.

Glucose measurements for a user are obtained (block 702). These glucose measurements are obtained from a glucose sensor of, for example, a continuous glucose level monitoring system with the glucose sensor being inserted at an insertion site of the user.

A duration of time during which an event or condition of the user that affects glucose levels of the user did not occur is detected (block 704). These events or conditions can be any of a variety of events or conditions, such as bouts of physical activity, meals consumed, sleep, and so forth.

One or more predicted glucose measurements are generated (block 706). The one or more predicted glucose measurements are glucose measurements that the user would have had if the event or condition had occurred. These predicted glucose measurements are a prediction of the impact of the event or condition on glucose of the user.

The predicted glucose measurements are caused to be displayed (block 708) or otherwise presented. Additionally or alternatively, the predicted glucose measurements can be communicated to or otherwise presented to a clinician, pharmacist, or other health care provider.

### Example System and Device

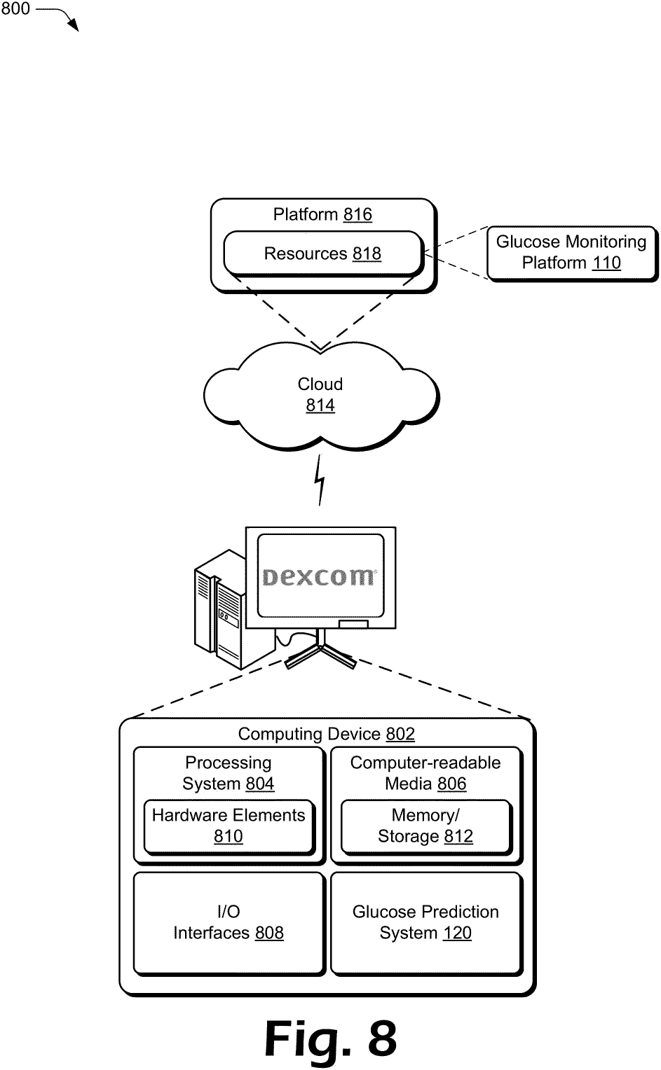

FIG. 8 illustrates an example of a system generally at 800 that includes an example of a computing device 802 that is representative of one or more computing systems and/or devices that may implement the various techniques described herein. This is illustrated through inclusion of the glucose prediction system 120. The computing device 802 may be, for example, a server of a service provider, a device associated with a client (e.g., a client device), an on-chip system, and/or any other suitable computing device or computing system.

The example computing device 802 as illustrated includes a processing system 804, one or more computer-readable media 806, and one or more I/O interfaces 808 that are communicatively coupled, one to another. Although not shown, the computing device 802 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 804 is representative of functionality to perform one or more operations using hardware. Accordingly, the processing system 804 is illustrated as including hardware elements 810 that may be configured as processors, functional blocks, and so forth. This may include implementation in hardware as an application specific integrated circuit or other logic device formed using one or more semiconductors. The hardware elements 810 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically-executable instructions.

The computer-readable media 806 is illustrated as including memory/storage 812. The memory/storage 812 represents memory/storage capacity associated with one or more computer-readable media. The memory/storage component 812 may include volatile media (such as random access memory (RAM)) and/or nonvolatile media (such as read only memory (ROM), Flash memory, optical disks, magnetic disks, and so forth). The memory/storage component 812 may include fixed media (e.g., RAM, ROM, a fixed hard drive, and so on) as well as removable media (e.g., Flash memory, a removable hard drive, an optical disc, and so forth). The computer-readable media 806 may be configured in a variety of other ways as further described below.

Input/output interface(s) 808 are representative of functionality to allow a user to enter commands and information to computing device 802, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 802 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

An implementation of the described modules and techniques may be stored on or transmitted across some form of computer-readable media. The computer-readable media may include a variety of media that may be accessed by the computing device 802. By way of example, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 802, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, communication media include wired media such as a wired network or direct-wired connection, and wireless media such as acoustic, RF, infrared, and other wireless media.

As previously described, hardware elements 810 and computer-readable media 806 are representative of modules, programmable device logic and/or fixed device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 810. The computing device 802 may be configured to implement particular instructions and/or functions corresponding to the software and/or hardware modules. Accordingly, implementation of a module that is executable by the computing device 802 as software may be achieved at least partially in hardware, e.g., through use of computer-readable storage media and/or hardware elements 810 of the processing system 804. The instructions and/or functions may be executable/operable by one or more articles of manufacture (for example, one or more computing devices 802 and/or processing systems 804) to implement techniques, modules, and examples described herein.

The techniques described herein may be supported by various configurations of the computing device 802 and are not limited to the specific examples of the techniques described herein. This functionality may also be implemented all or in part through use of a distributed system, such as over a “cloud” 814 via a platform 816 as described below.

The cloud 814 includes and/or is representative of a platform 816 for resources 818. The platform 816 abstracts underlying functionality of hardware (e.g., servers) and software resources of the cloud 814. The resources 818 may include applications and/or data that can be utilized while computer processing is executed on servers that are remote from the computing device 802. Resources 818 can also include services provided over the Internet and/or through a subscriber network, such as a cellular or Wi-Fi network.

The platform 816 may abstract resources and functions to connect the computing device 802 with other computing devices. The platform 816 may also serve to abstract scaling of resources to provide a corresponding level of scale to encountered demand for the resources 818 that are implemented via the platform 816. Accordingly, in an interconnected device embodiment, implementation of functionality described herein may be distributed throughout the system 800. For example, the functionality may be implemented in part on the computing device 802 as well as via the platform 816 that abstracts the functionality of the cloud 814.

In some aspects, the techniques described herein relate to a method implemented in a continuous glucose level monitoring system, the method including: obtaining, from a glucose sensor of the continuous glucose level monitoring system, glucose measurements measured for a user, the glucose sensor being inserted at an insertion site of the user; detecting a bout of physical activity performed by the user; predicting an impact of the physical activity on glucose of the user by generating, based on the glucose measurements, one or more predicted glucose measurements that the user would have had if, during a time the user was performing the bout of physical activity, the user had not performed the bout of physical activity; and causing the one or more predicted glucose measurements to be displayed.

In some aspects, the techniques described herein relate to a method, further including identifying a subset of the glucose measurements that were measured immediately preceding the bout of physical activity, and the generating including generating the one or more predicted glucose measurements based on the subset of glucose measurements.

In some aspects, the techniques described herein relate to a method, wherein the generating includes generating the one or more predicted glucose measurements using a machine learning system trained to predict glucose measurements for the user based on previous glucose measurements of the user.

In some aspects, the techniques described herein relate to a method, wherein the generating includes generating the one or more predicted glucose measurements based on one or more of physiological parameters of the user, demographic information of the user, or clinical information of the user.

In some aspects, the techniques described herein relate to a method, wherein the generating includes generating the one or more predicted glucose measurements using a physiological or phenomenological model.

In some aspects, the techniques described herein relate to a method, wherein the generating includes generating predicted glucose measurements for a duration of the bout of physical activity.

In some aspects, the techniques described herein relate to a method, wherein the generating further includes generating predicted glucose measurements for a duration of time after the bout of physical activity.

In some aspects, the techniques described herein relate to a method, wherein the detecting a bout of physical activity includes detecting, as a bout of physical activity, a duration of time during which the user took at least a threshold number of steps per minute for at least a threshold amount of time without dropping below the threshold number of steps for at least a consecutive number of minutes.

In some aspects, the techniques described herein relate to a method, wherein the detecting a bout of physical activity includes detecting, as a bout of physical activity, a duration of time during which a heart-rate based intensity value of the user exceeded a threshold amount for at least a threshold amount of time without dropping below the threshold amount for at least a consecutive amount of time.

In some aspects, the techniques described herein relate to a method, wherein the detecting a bout of physical activity includes detecting, as a bout of physical activity, a duration of time during which a number of Metabolic Equivalents value of the user exceeded a threshold amount for at least a threshold amount of time without dropping below the threshold amount for at least a consecutive amount of time.

In some aspects, the techniques described herein relate to a device including a diabetes management monitoring system, the device including: a biological data detection module, implemented at least in part in hardware, to obtain, from a sensor of the diabetes management monitoring system, glucose measurements measured for a user; an activity detection module, implemented at least in part in hardware, to detect a bout of physical activity performed by the user; a glucose prediction module, implemented at least in part in hardware, to predict an impact of the physical activity on glucose of the user by generating, based on the glucose measurements, one or more predicted glucose measurements that the user would have had if, during a time the user was performing the bout of physical activity, the user had not performed the bout of physical activity; a user interface module, implemented at least in part in hardware, to cause the one or more predicted glucose measurements to be displayed.

In some aspects, the techniques described herein relate to a device, wherein the glucose prediction module is further to identify a subset of the glucose measurements that were measured immediately preceding the bout of physical activity and generate the one or more predicted glucose measurements based on the subset of glucose measurements.

In some aspects, the techniques described herein relate to a device, wherein the glucose prediction module is further to generate the one or more predicted glucose measurements using a machine learning system trained to predict glucose measurements for the user based on previous glucose measurements of the user.

In some aspects, the techniques described herein relate to a device, wherein the glucose prediction module is further to generate predicted glucose measurements for a duration of the bout of physical activity.

In some aspects, the techniques described herein relate to a device, wherein the activity detection module is further to detect, as a bout of physical activity, a duration of time during which the user took at least a threshold number of steps per minute for at least a threshold amount of time without dropping below the threshold number of steps for at least a consecutive number of minutes.

In some aspects, the techniques described herein relate to a method implemented in a diabetes management monitoring system, the method including: obtaining, from a sensor of the diabetes management monitoring system, glucose measurements measured for a user; detecting biological data of the user indicating the user is highly stressed for a duration of time; predicting an impact of the biological data on glucose of the user by generating, based on the glucose measurements, one or more predicted glucose measurements that the user would have had if, during the duration of time, the user had not been highly stressed for the duration of time; and causing the one or more predicted glucose measurements to be displayed.

In some aspects, the techniques described herein relate to a method implemented in a diabetes management monitoring system, the method including: obtaining, from a sensor of the diabetes management monitoring system, glucose measurements measured for a user; determining that a bout of physical activity was not performed by the user during a duration of time; predicting an impact of not performing the physical activity on glucose of the user by generating, based on the glucose measurements, one or more predicted glucose measurements that the user would have had if, during a time the user was performing the bout of physical activity, the user had performed the bout of physical activity; and causing the one or more predicted glucose measurements to be displayed.

In some aspects, the techniques described herein relate to a method implemented in a diabetes management monitoring system, the method including: obtaining, from a sensor of the diabetes management monitoring system, glucose measurements measured for a user; detecting an event or condition of the user that affects glucose levels of the user; predicting an impact of the event or condition on glucose of the user by generating, based on the glucose measurements, one or more predicted glucose measurements that the user would have had if the event or condition had not occurred; and causing the one or more predicted glucose measurements to be displayed.

In some aspects, the techniques described herein relate to a method, wherein the event or condition includes food or drink consumed by the user.

In some aspects, the techniques described herein relate to a method, wherein the event or condition includes times when the user is sleeping or a sleep state of the user.

In some aspects, the techniques described herein relate to a method, wherein the event or condition includes medicine taken by the user.

In some aspects, the techniques described herein relate to a method, wherein the event or condition includes user engagement with a glucose monitoring application.

### Conclusion

Although the systems and techniques have been described in language specific to structural features and/or methodological acts, it is to be understood that the systems and techniques defined in the appended claims are not necessarily limited to the specific features or acts described. Rather, the specific features and acts are disclosed as example forms of implementing the claimed subject matter.

What is claimed is:

1. A method implemented in a continuous glucose level monitoring system, the method comprising:
obtaining, from a glucose sensor of the continuous glucose level monitoring system, glucose measurements measured for a user, the glucose sensor being inserted at an insertion site of the user;
detecting a bout of physical activity performed by the user;
predicting an impact of the physical activity on glucose of the user by generating, based on the glucose measurements, one or more predicted glucose measurements that the user would have had if, during a time the user was performing the bout of physical activity, the user had not performed the bout of physical activity; and
causing the one or more predicted glucose measurements to be displayed.

2. The method of claim 1, further comprising identifying a subset of the glucose measurements that were measured immediately preceding the bout of physical activity, and the generating including generating the one or more predicted glucose measurements based on the subset of glucose measurements.

3. The method of claim 1, wherein the generating includes generating the one or more predicted glucose measurements using a machine learning system trained to predict glucose measurements for the user based on previous glucose measurements of the user.

4. The method of claim 1, wherein the generating includes generating the one or more predicted glucose measurements based on one or more of physiological parameters of the user, demographic information of the user, or clinical information of the user.

5. The method of claim 1, wherein the generating includes generating the one or more predicted glucose measurements using a physiological or phenomenological model.

6. The method of claim 1, wherein the generating includes generating predicted glucose measurements for a duration of the bout of physical activity.

7. The method of claim 6, wherein the generating further includes generating predicted glucose measurements for a duration of time after the bout of physical activity.

8. The method of claim 1, wherein the detecting a bout of physical activity includes detecting, as a bout of physical activity, a duration of time during which the user took at least a threshold number of steps per minute for at least a threshold amount of time without dropping below the threshold number of steps for at least a consecutive number of minutes.

9. The method of claim 1, wherein the detecting a bout of physical activity includes detecting, as a bout of physical activity, a duration of time during which a heart-rate based intensity value of the user exceeded a threshold amount for at least a threshold amount of time without dropping below the threshold amount for at least a consecutive amount of time.

10. The method of claim 1, wherein the detecting a bout of physical activity includes detecting, as a bout of physical activity, a duration of time during which a number of Metabolic Equivalents value of the user exceeded a threshold amount for at least a threshold amount of time without dropping below the threshold amount for at least a consecutive amount of time.

11. A device including a diabetes management monitoring system, the device comprising:
a biological data detection module, implemented at least in part in hardware, to obtain, from a sensor of the diabetes management monitoring system, glucose measurements measured for a user;
an activity detection module, implemented at least in part in hardware, to detect a bout of physical activity performed by the user;
a glucose prediction module, implemented at least in part in hardware, to predict an impact of the physical activity on glucose of the user by generating, based on the glucose measurements, one or more predicted glucose measurements that the user would have had if, during a time the user was performing the bout of physical activity, the user had not performed the bout of physical activity; and
a user interface module, implemented at least in part in hardware, to cause the one or more predicted glucose measurements to be displayed.

12. The device of claim 11, wherein the glucose prediction module is further to identify a subset of the glucose measurements that were measured immediately preceding the bout of physical activity and generate the one or more predicted glucose measurements based on the subset of glucose measurements.

13. The device of claim 11, wherein the glucose prediction module is further to generate the one or more predicted glucose measurements using a machine learning system trained to predict glucose measurements for the user based on previous glucose measurements of the user.

14. The device of claim 11, wherein the glucose prediction module is further to generate predicted glucose measurements for a duration of the bout of physical activity.

15. The device of claim 11, wherein the activity detection module is further to detect, as a bout of physical activity, a duration of time during which the user took at least a threshold number of steps per minute for at least a threshold amount of time without dropping below the threshold number of steps for at least a consecutive number of minutes.

16. A method implemented in a diabetes management monitoring system, the method comprising:
obtaining, from a sensor of the diabetes management monitoring system, glucose measurements measured for a user;
detecting an event or condition of the user that affects glucose levels of the user;
predicting an impact of the event or condition on glucose of the user by generating, based on the glucose measurements, one or more predicted glucose measurements that the user would have had if the event or condition had not occurred; and
causing the one or more predicted glucose measurements to be displayed.

17. The method of claim 16, wherein the event or condition includes food or drink consumed by the user.

18. The method of claim 16, wherein the event or condition includes times when the user is sleeping or a sleep state of the user.

19. The method of claim 16, wherein the event or condition includes medicine taken by the user.

20. The method of claim 16, wherein the event or condition includes user engagement with a glucose monitoring application.
